(19) United States
(12) Patent Application Publication
(10) Pub. No.: US 2010/0224191 A1
(43) Pub. Date: Sep. 9, 2010

(54) Automated Oxygen Delivery System

(75) Inventors: Paul Dixon, London (GB); Thomas Westfall, Riverside, CA (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(73) Assignee: CARDINAL HEALTH 207, INC., Yorba Linda, CA (US)

(21) Appl. No.: 12/399,341

(22) Filed: Mar. 6, 2009

(51) Int. Cl. A61M 16/12

(52) U.S. Cl. 128/204.23

(57) ABSTRACT

The present invention advantageously provides a system for automatically delivering oxygen to a patient, including a sensor to measure an amount of oxygen in a bloodstream of a patient, a pneumatics subsystem and a control subsystem. The pneumatics subsystem includes an oxygen inlet, an air inlet, a gas mixture outlet, and a gas delivery mechanism to blend the oxygen and air to form a gas mixture having a delivered oxygen concentration, and to deliver the gas mixture to the patient. The control subsystem includes an input device to receive a desired concentration of oxygen in the bloodstream of the patient, a sensor interface to receive measurement data and status information associated with the measurement data from the sensor, a pneumatics subsystem interface to send commands to, and receive data from, the pneumatics subsystem, and a processor to control the delivered oxygen concentration based on the desired oxygen concentration, the measurement data and the status information.

### FIELD OF THE INVENTION

The present invention is generally directed to oxygen delivery systems and methods. More particularly, the present invention is directed to an automated oxygen delivery system.

### BACKGROUND OF THE INVENTION

Many patients require respiratory support, including additional oxygen and/or assisted ventilation. Infants, particularly those born before term, may be unable to maintain adequate respiration and require support in the form of a breathing gas mixture combined with ventilatory assistance. The breathing gas mixture has an elevated fraction of oxygen (FiO₂) compared to room air, while the ventilatory assistance provides elevated pressure at the upper airway. A significant number of infants receiving respiratory support will exhibit episodes of reduced blood oxygen saturation, or desaturation, i.e., periods in which oxygen uptake in the lungs is inadequate and blood oxygen saturation falls. These episodes may occur as frequently as twenty times per hour, and each episode must be carefully managed by the attending health care professional.

Most prior art systems require the attendant to monitor the blood oxygen saturation and manually adjust the ventilator settings to provide additional oxygen as soon as desaturation is detected. Similarly, the attendant must reduce the oxygen delivered to the patient once the blood oxygen saturation has been restored to a normal range. Failure to provide additional oxygen rapidly to the patient can lead to hypoxic ischemic damage, including neurological impairment, and, if prolonged, may cause death. Similarly, failure to reduce the oxygen delivered to the patient following recovery also has clinical sequelae, the most frequent of which is Retinopathy of Prematurity, a form of blindness caused by oxidation of the optical sensory neurons. While at least one prior art system has attempted to close a control loop around delivered FiO₂ by using measured arterial hemoglobin oxygen saturation levels in the patient, this system does not safely and adequately detect and accommodate invalid measurement data, placing the patient at risk for at least those conditions noted above.

Accordingly, an improved oxygen delivery system is needed that automatically and safely controls the amount of oxygen delivered to a patient based on the amount of oxygen that is measured in the bloodstream and the status information associated with the measurement.

### SUMMARY OF THE INVENTION

Embodiments of the present invention advantageously provide a system for automatically delivering oxygen to a patient.

In one embodiment, an automated oxygen delivery system includes a sensor to measure an amount of oxygen in a bloodstream of a patient, a pneumatics subsystem and a control subsystem. The pneumatics subsystem includes an oxygen inlet, an air inlet, a gas mixture outlet, and a gas delivery mechanism to blend the oxygen and air to form a gas mixture having a delivered oxygen concentration, and to deliver the gas mixture to the patient. The control subsystem includes an input device to receive a desired concentration of oxygen in the bloodstream of the patient, a sensor interface to receive measurement data and status information associated with the measurement data from the sensor, a pneumatics subsystem interface to send commands to, and receive data from, the pneumatics subsystem, and a processor to control the delivered oxygen concentration based on the desired oxygen concentration, the measurement data and the status information.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a block diagram of an automated oxygen delivery system, in accordance with an embodiment of the present invention.

FIG. 2A is a block diagram of a gas delivery mechanism, in accordance with an embodiment of the present invention.

FIG. 2B is a block diagram of a gas delivery mechanism, in accordance with another embodiment of the present invention.

FIG. 3 is a control process diagram for an automated oxygen delivery system, in accordance with an embodiment of the present invention.

FIG. 4 is flow chart depicting a method for automatically delivering oxygen to a patient, in accordance with an embodiment of the present invention.

FIG. 5 is flow chart depicting a method for automatically delivering oxygen to a patient, in accordance with another embodiment of the present invention.

### DETAILED DESCRIPTION OF THE INVENTION

The invention will now be described with reference to the drawing figures, in which like reference numerals refer to like parts throughout.

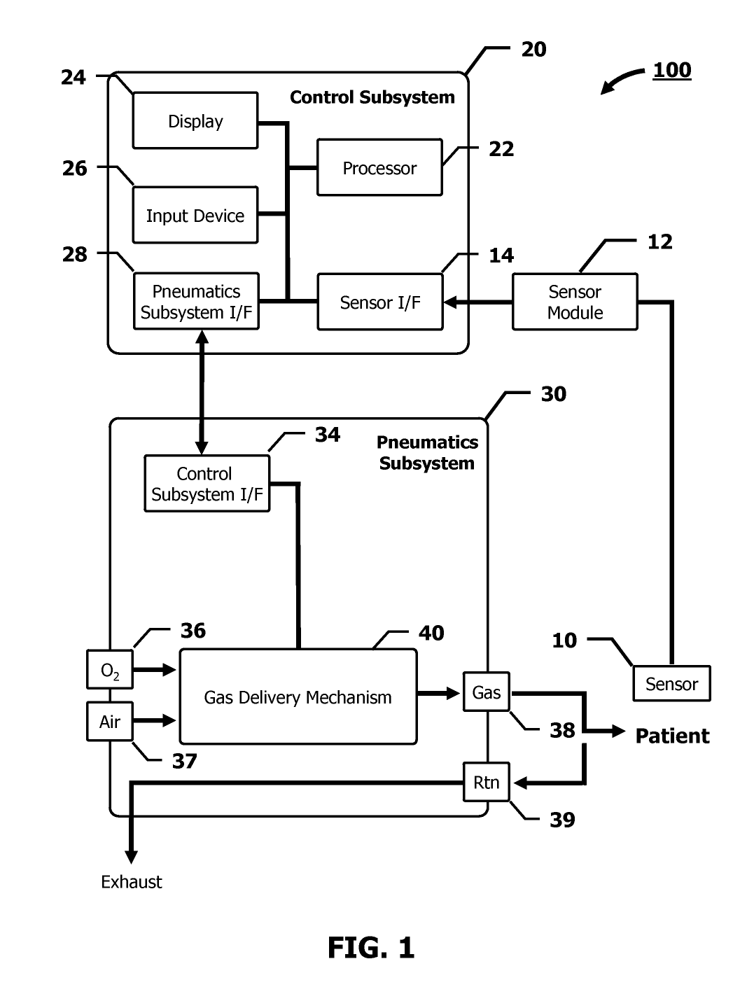

FIG. 1 is a block diagram of an automated oxygen delivery system, in accordance with an embodiment of the present invention. Generally, automated oxygen delivery system 100 is a software-driven, servo-controlled gas delivery system that provides a full range of volume and pressure ventilation for neonatal, pediatric and adult patients. More specifically, automated oxygen delivery system 100 safely maintains the amount of oxygen measured in the patient's bloodstream within a user-selectable range by titrating the FiO₂ based on the oxygen measurements. As depicted in FIG. 1, automated oxygen delivery system 100 includes a sensor 10 that measures the amount of oxygen in the bloodstream of the patient, a control subsystem 20 and a pneumatics subsystem 30.

In a preferred embodiment, sensor 10 is a Masimo Signal Extraction pulse oximeter sensor (Masimo Corporation, Irvine, Calif.) that measures the absorption of light in two different wavelengths, such as red and infrared light, from which that fraction of the red blood cells in the optical pathway that are carrying oxygen, and hence the amount of oxygen in the patient's bloodstream, can be determined. In this embodiment, sensor module 12 is a Masimo interface board, such as the MS-11, MS-13, etc., sensor 10 is an Masimo pulse oximeter sensor, such as the LNCS (or LNOP) Adtx, Pdtx, Inf, Neo, NeoPt, etc., that is coupled to control subsystem 20 though sensor module 12 and attendant interface cables. Sensor module 12 includes a microcontroller, digital signal processor and supporting circuitry to drive the active components within sensor 10, such as red and infrared LEDs, capture the light signals generated by sensor 10, process these signals, and generate measurement data and status information associated with the sensor. Sensor module 12 calculates the saturation of peripheral oxygen, S_{P}O₂, in the bloodstream of the patient and the pulse rate of the patient based on these light signals, generates status information associated with the S_{P}O₂ data, including, for example, a perfusion index, a signal quality index, etc., and communicates this data to control subsystem 20 through sensor interface 14, such as an RS-232 serial interface. Alternatively, sensor module 12 may be incorporated within control subsystem 20 itself, replacing sensor interface 14.

In this embodiment, the perfusion index is the fractional variation in the optical absorption of oxygenated red blood cells between the systole and diastole periods of an arterial pulse. The signal quality index generally provides a confidence metric for the S_{P}O₂, and, in this pulse oximeter embodiment, the signal quality index is based on variations in the optical absorption related to, and not related to, the cardiac cycle. Additionally, sensor module 12 may identify measurement artifacts or sensor failures, such as optical interference (e.g., too much ambient light), electrical interference, sensor not detected, sensor not attached, etc., and provide this status information to control subsystem 20. In an alternative embodiment, sensor module 12 may provide red and infrared plethysmorgraphic signals directly to sensor interface 14 at a particular sample resolution and sample rate, such as, for example, 4 bytes/signal and 60 Hz, from which the S_{P}O₂ is calculated directly by control subsystem 20. These signals may be processed, averaged, filtered, etc., as appropriate, and used to generate the perfusion index, the signal quality index, various signal metrics, etc.

In another embodiment, sensor 10 is a transcutaneous gas tension sensor, such as, for example, a Radiometer TCM 4 or TCM40 transcutaneous monitor (Radiometer Medical ApS, Bronshoj, Denmark), that directly measures the partial pressure of oxygen in arteriolar blood, i.e., the blood in the surface capillary blood vessels, using a gas permeable membrane placed in close contact with skin. The membrane is heated to between 38° C. and 40° C. to encourage the surface blood vessels to dilate, and oxygen diffuses through the skin surface and the permeable membrane until the oxygen partial pressure inside the sensor is in equilibrium with the oxygen partial pressure in the blood. The transcutaneous gas tension sensor includes electrochemical cells, with silver and platinum electrodes and a reservoir of dissolved chemicals, that directly detect oxygen as well as carbon dioxide in solution in the blood. The measurement data provided by this sensor include arterial oxygen partial pressure measurement, PtcO₂, and arterial carbon dioxide partial pressure measurement, PtcCO₂, while status information may include heat output, sensor temperature, and skin perfusion. These data may be supplemented by additional information acquired by a pulse oximeter. In this transcutaneous gas tension embodiment, sensor module 12 may be provided as an independent module, or as a component within control subsystem 20.

In yet another embodiment, sensor 10 is an invasive catheter blood analyzer, such as, for example, a Diametric Neocath, Paratrend or Neotrend intra-arterial monitor, that is inserted into a blood vessel and directly measures various chemical constituents of the blood, such as O₂, CO₂, pH, etc., using chemoluminescent materials which either produce, or absorb, particular wavelengths of light depending the quantity of dissolved molecules in proximity to the sensor. The light is then transmitted along an optical fiber in the catheter to an external monitor device, such as sensor module 12. The measurement data provided by this sensor include dissolved oxygen in the blood, pO₂, dissolved carbon dioxide in the blood, pCO₂, blood acidity pH, and blood temperature. In this invasive catheter blood analyzer embodiment, sensor module 12 may be provided as an independent module or as a component within control subsystem 20.

Control subsystem 20 controls all of the ventilator functions, sensor measurement processing, gas calculations, monitoring and user interface functions. In a preferred embodiment, control subsystem 20 includes, inter alia, display 24, one or more input device(s) 26, sensor interface 14, pneumatics subsystem interface 28 and one or more processor(s) 22 coupled thereto. For example, display 24 may be a 12.1-inch, 800×600 backlit, active matrix liquid crystal display (LCD), that presents the graphical user interface (GUI) to the user, which includes all of the adjustable controls and alarms, as well as displays waveforms, loops, digital monitors and alarm status. Input devices 26 may include an analog resistive touch screen overlay for display 24, a set of membrane key panel(s), an optical encoder, etc. Software, executed by processor 22, cooperates with the touch screen overlay to provide a set of context sensitive soft keys to the user, while the membrane key panel provides a set of hard keys for dedicated functions. For example, the user may select a function with a soft key and adjust a particular setting using the optical encoder, which is accepted or canceled by pressing an appropriate hard key. Pneumatics subsystem interface 28 is coupled to control subsystem interface 34, disposed in pneumatics subsystem 30, to send commands to, and receive data from, the pneumatics subsystem 30 over a high-speed serial channel, for example.

Processor 22 generally controls the delivered oxygen concentration to the patient based on the desired arterial oxygen concentration, input by the user, and the measurement data and status information received from sensor 10. For example, processor 22 performs gas calculations, controls all valves, solenoids, and pneumatics subsystem electronics required to deliver blended gas to the patient. Additionally, processor 22 manages the GUI, including updating display 24, monitoring the membrane keypad, analog resistive touch screen, and optical encoder for activity. The gas control processes executed by processor 22 are discussed in more detail below.

Pneumatics subsystem 30 contains all of the mechanical valves, sensors, microcontrollers, analog electronics, power supply, etc., to receive, process and deliver the gas mixture to the patient. In a preferred embodiment, pneumatics subsystem 30 includes, inter alia, control subsystem interface 34, one or more optional microcontrollers (not shown), oxygen inlet 36, air inlet 37, gas mixture outlet 38, an optional exhalation inlet 39, and gas delivery mechanism 40, which blends the oxygen and air to form a gas mixture having a delivered oxygen concentration, and then delivers the gas mixture to the patient through gas mixture outlet 38. In one embodiment, pneumatics subsystem 30 receives oxygen through oxygen inlet 36 and high-pressure air through air inlet 37, filters and blends these gases through a gas blender, and then delivers the appropriate pressure or volume of the gas mixture through gas mixture outlet 38. In another embodiment, pneumatics subsystem 30 receives oxygen through oxygen inlet 36 and high-pressure air through air inlet 37, filters these gases, and then delivers the a calculated flow rate of air and a calculated flow rate of oxygen to the patient outlet such as to provide the appropriate pressure or volume of gas mixture with the required fraction of oxygen FiO2 through gas mixture outlet 38. In a further embodiment, pneumatics subsystem 30 receives oxygen pre-mixed with an alternate gas, such as nitrogen, helium, 80/20 heliox, etc., through air inlet 37, and control subsystem 30 adjusts blending, volume delivery, volume monitoring and alarming, as well as FiO₂ monitoring and alarming, based on the properties of the air/alternate gas inlet supply. A heated expiratory system, nebulizer, and compressor may also be provided.

In one embodiment, control subsystem 20 and pneumatics subsystem 30 are respectively accommodated within separate physical modules or housings, while in another embodiment, control subsystem 20 and pneumatics subsystem 30 are accommodated within a single module or housing.

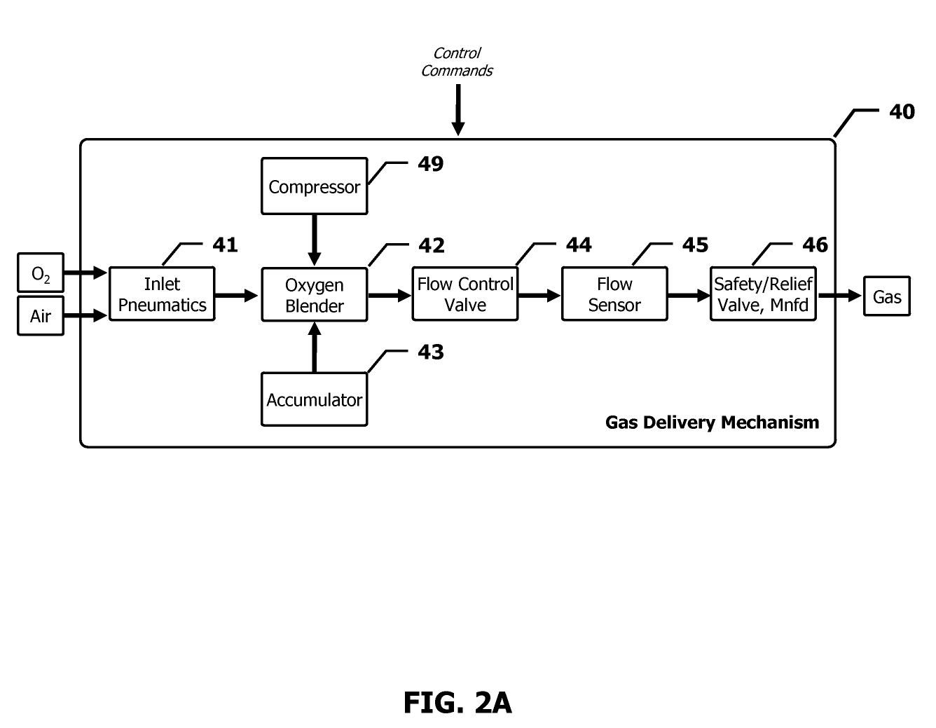

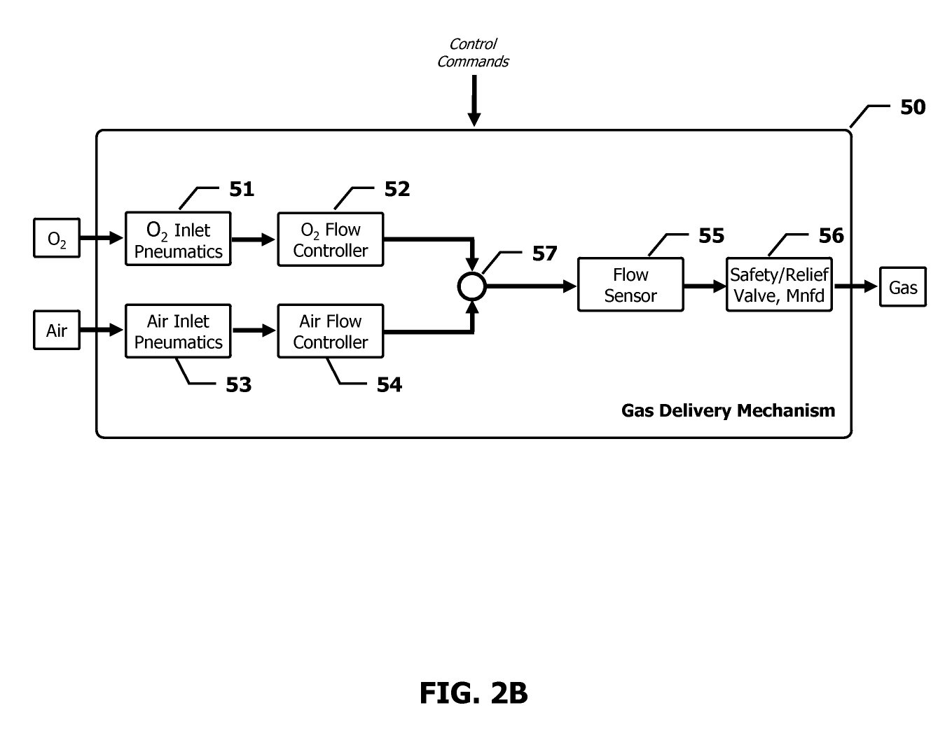

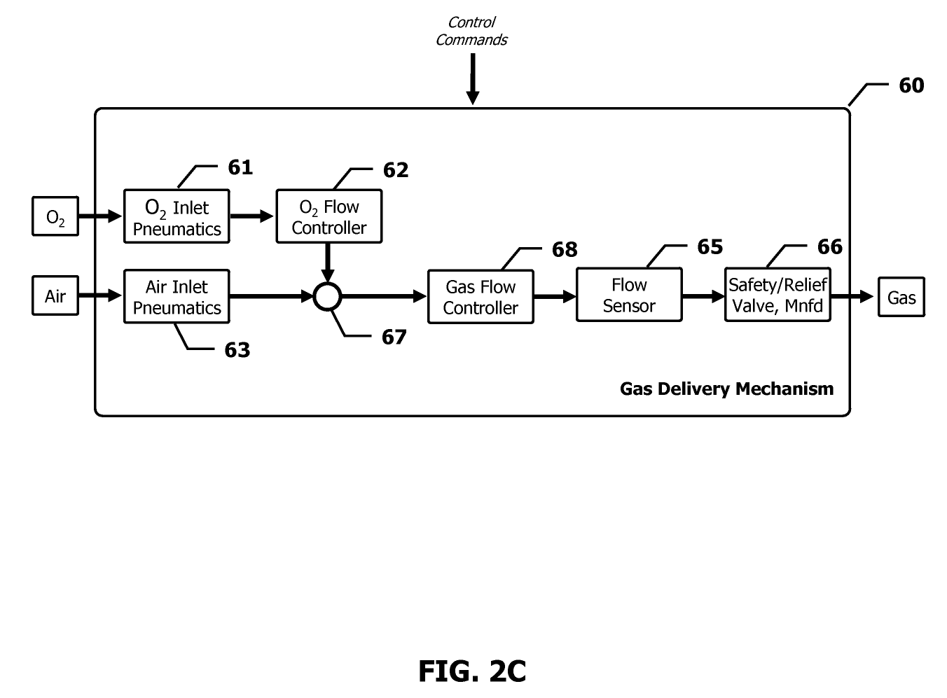

FIG. 2A is a block diagram of a gas delivery mechanism, in accordance with an embodiment of the present invention. In this embodiment, gas delivery mechanism 40 includes, inter alia, inlet pneumatics 41, oxygen blender 42, accumulator system 43, flow control valve 44, flow control sensor 45, and safety/relief valve and outlet manifold 46. In one embodiment, compressor 49 provides supplemental or replacement air to oxygen blender 42. Inlet pneumatics 41 receives clean O₂ and air, or an air/alternate gas mixture, provides additional filtration, and regulates the O₂ and the air for delivery to oxygen blender 42, which mixes the O₂ and the air to the desired concentration as determined by commands received from the control subsystem 20. Accumulator system 43 provides peak flow capacity. Flow control valve 44 generally controls the flow rate of the gas mixture to the patient, and the flow sensor 45 provides information about the actual inspiratory flow to the control subsystem 20. The gas is delivered to the patient through safety/relief valve and outlet manifold 46.

In one embodiment, inlet pneumatics 41 includes a manifold with region or country specific “smart” fittings for high-pressure (e.g., 20 to 80 psig) air and oxygen, sub-micron inlet filters that remove aerosol and particulate contaminants from the inlet gas, pressure transducers that detect a loss of each inlet gas, a check valve on the air inlet, and a pilot oxygen switch on the oxygen inlet. The oxygen switch acts as both an oxygen shut off valve when no power is applied, and a check valve when power is applied. A downstream air regulator and O₂ relay combination may also be used to provide balanced supply pressure to the gas blending system. The air regulator reduces the air supply pressure to 11.1 PSIG and pilots the O₂ relay to track at this pressure. When compressor 49 is provided, the air supply pressure is regulated from about 5 PSIG to about 10 PSIG, or, preferably, from about 6 PSIG to about 9.5 PSIG.

When supply air pressure falls below about 20 PSIG, compressor 49 is activated to automatically supply air to the oxygen blender 42. When compressor 49 is not provided, the crossover solenoid opens to deliver high-pressure oxygen to the air regulator, allowing the air regulator to supply regulated O₂ pressure to pilot the O₂ relay. Additionally, oxygen blender 42 simultaneously moves to a 100% O₂ position, so that full flow to the patient is maintained. Similarly, when oxygen pressure falls below about 20 PSIG, the crossover solenoid stays closed, the oxygen switch solenoid is de-energized, the blender moves to 21% O₂, and the regulated air pressure provides 100% air to oxygen blender 42.

Oxygen blender 42 receives the supply gases from the inlet pneumatics 41 and blends the two gases to a particular value provided by control subsystem 20. In one embodiment, oxygen blender 42 includes a valve, stepper motor, and drive electronics.

Accumulator 43 is connected to the outlet manifold of oxygen blender 42 using a large-orifice piloted valve, in parallel with a check-valve. Accumulator 43 stores blended gas from oxygen blender 42, which increases system efficiency, and provides the breath-by-breath tidal volume and peak flow capacity at relatively lower pressure, advantageously resulting in lower system power requirements. Accumulator gas pressure cycles between about 2 PSIG and about 12 PSIG, depending on the tidal volume and peak flow requirements. An accumulator bleed orifice allows approximately 6 liters/min of gas to exit the accumulator, thereby providing a stable O₂ mix even with no flow from the flow control valve. A pressure relief valve provides protection from pressure in excess of about 12 PSIG. A water dump solenoid may be activated periodically, for a predetermined period of time, to release a respective amount of gas from accumulator 43 in order to purge any moisture that may have accumulated. A regulator is attached just down stream of the accumulator to provide a regulated pressure source for the pneumatics. A bleed flow of approximately 0.1 liter/min is sampled by an O₂ sensor to measure the delivered FiO₂. In another embodiment, accumulator 43 may be omitted from gas delivery mechanism 40.

A flow control system provides the desired flow rate of gas mixture to the patient, and includes flow control valve 44 and flow sensor 45, as well as a gas temperature sensor and circuit pressure sensors. The high-pressure gas stored in accumulator 43 feeds flow control valve 44, which is controlled by control subsystem 20 via control subsystem interface 34. Flow sensor 45, along with the gas temperature sensor and the circuit pressure sensors, provide feedback to control subsystem 20. Periodically, control subsystem 20 reads the sensors, calculates and provides a position command to flow control valve 44. Control subsystem 20 adjusts for flow, gas temperature, gas density, and backpressure. The flow proportional pressure drop is measured with a pressure transducer, suitably nulled using one or more auto zero solenoids. Importantly, when the patient is a neonate, the check/bypass valve is closed, and the gas mixture continues to flow from oxygen blender 42 to accumulator 43 to provide the required minimum blender flow, but the gas mixture does not flow back from accumulator 43 to the patient circuit. This advantageously minimizes the time taken for a change in set oxygen fraction to reach the patient outlet.

Safety/relief valve and outlet manifold 46 includes, inter alia, a three way safety solenoid, a piloted sub ambient/over pressure relief valve, and a check valve. Safety/relief valve and manifold 46 prevents over-pressure in the breathing circuit, and allows the patient to breath ambient air during a “safety valve open” alarm. A safe state can also be activated due to a complete loss of gas supplies or complete loss of power. The pressure relief valve is a mechanical relief valve that will not allow pressure to exceed a certain value with a maximum gas flow of about 150 liter/min. The sub ambient valve is piloted with the safety solenoid and on loss of power or a “vent inop” the safety solenoid will be deactivated, which causes the sub ambient valve to open allowing the patient to breath ambient gas. In this case, the check valve helps to insure that the patient will inspire from the sub ambient valve and expire through the exhalation valve thus not rebreathing patient gas.

In a preferred embodiment, the delivered gas is forced into the patient by closing a servo-controlled exhalation valve. The patient is allowed to exhale by the exhalation valve, which also maintains baseline pressure or PEEP. The exhaled gas exits the patient through the expiratory limb of the patient circuit and, in one embodiment, re-enters pneumatics subsystem 30 through exhalation inlet 39, passes through a heated expiratory filter to an external flow sensor, and then out through an exhalation valve to ambient air.

Advantageously, the gas volume may be monitored at the expiratory limb of the machine or at the patient wye, which allows for more accurate patient monitoring, particularly in infants, while allowing the convenience of an expiratory limb flow sensor protected by a heated filter that is preferred in the adult ICU. And, both tracheal and esophageal pressure may be measured. An optional CO₂ sensor, such as, for example, a Novametrix Capnostat 5 Mainstream CO₂ sensor, may be attached to the breathing circuit at the patient wye, connecting to the control subsystem 20 through a serial communications port, to provide monitoring of the end-tidal pressure of the exhaled CO₂ and the exhaled CO₂ pressure waveform. When used in conjunction with a wye flow sensor, or when breathing circuit compliance compensation is enabled, the CO₂ pressure waveform may also be used to derive secondary monitors.

FIG. 2B is a block diagram of a gas delivery mechanism, in accordance with another embodiment of the present invention. In this embodiment, gas delivery mechanism 50 includes, inter alia, oxygen inlet pneumatics 51, oxygen flow controller 52, air inlet pneumatics 53, air flow controller 54, gas mixing manifold 57, flow control sensor 55, and safety/relief valve and outlet manifold 56. Oxygen inlet pneumatics 51 receives clean O₂, provides additional filtration, and provides the O₂ to oxygen flow controller 52. Air inlet pneumatics 53 receives clean air, or an air/alternate gas mixture, provides additional filtration, and provides the air to air flow controller 54. In one embodiment, air flow controller 54 is a servo-controlled flow control valve, while in another embodiment, air flow controller 54 is a variable-speed blower or pump. The oxygen flow controller 52 and the air flow controller 54 control the respective flow of oxygen and air supplied to gas mixing manifold 57 in strict ratio, as determined by commands received from the control subsystem 20. The flow sensor 55 provides information about the actual inspiratory flow to the control subsystem 20, and the gas is delivered to the patient through safety/relief valve and outlet manifold 56. In this embodiment, the oxygen ratio of the delivered gas mixture depends upon the controlled flow rates of oxygen and air (Q_{oxygen} and Qₐᵢᵣ, respectively), as given by Equation (1):

$$\% {O}_{2} = \frac{( 100 * Qoxygen + 21 * Qair )}{( Qoxygen + Qair )} = 21 + 79 * \frac{Qoxygen}{( Qoxygen + Qair )} \qquad ( 1 )$$

FIG. 2C is a block diagram of a gas delivery mechanism, in accordance with yet another embodiment of the present invention. In this embodiment, gas delivery mechanism 60 includes, inter alia, oxygen inlet pneumatics 61, oxygen flow controller 62, air inlet pneumatics 63, gas mixing manifold 67, gas flow controller 68, flow control sensor 65, and safety/relief valve and outlet manifold 66. Air inlet pneumatics 63 receives clean air, or an air/alternate gas mixture, provides additional filtration, and provides the air to gas mixing manifold 67. Oxygen inlet pneumatics 61 receives clean 02, provides additional filtration, and provides the O₂ to oxygen flow controller 62, which controls the flow of oxygen supplied to gas mixing manifold 67, as determined by commands received from the control subsystem 20. The mixed gas is then provided to gas flow controller 68, which controls the flow of mixed gas supplied to the patient, as determined by commands received from the control subsystem 20. In a preferred embodiment, gas flow controller 68 is a variable-speed blower or pump. The flow sensor 65 provides information about the actual inspiratory flow to the control subsystem 20, and the gas is delivered to the patient through safety/relief valve and outlet manifold 66. In this embodiment, the oxygen ratio of the delivered gas mixture depends upon the controlled flow rates of oxygen and mixed gas (Q_{oxygen} and Q_{gas}, respectively), as given by Equation (2):

$$\% {O}_{2} = \frac{( \begin{matrix}100 * Qoxygen + \\ 21 * ( Qgas - Qoxygen )\end{matrix} )}{Qgas} = 21 + 79 * \frac{Qoxygen}{Qgas} \qquad ( 2 )$$

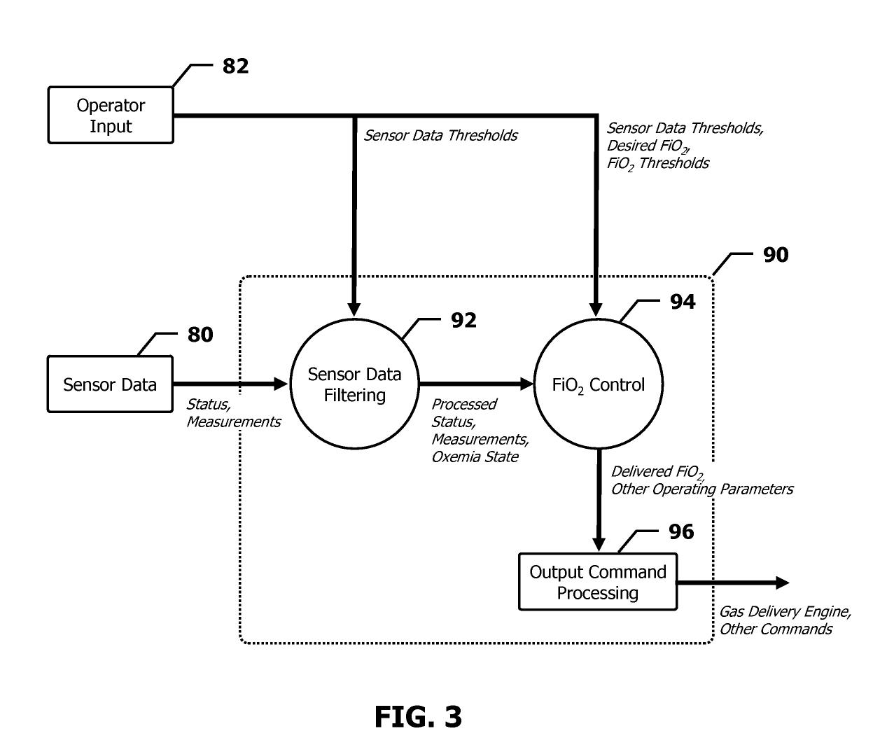

FIG. 3 is a control process diagram for an automated oxygen delivery system, in accordance with an embodiment of the present invention. Generally, automated oxygen delivery system 100 controls delivered FiO₂ to the patient, in a closed-loop fashion, based on the measurements of the oxygen concentration in the patient's bloodstream and the desired oxygen concentration provided by a user. Closed-loop FiO₂ control process 90 is embodied by software and/or firmware executed by one or more processor(s) 22, and receives operator input 82 via input device(s) 26, receives sensor data 80 from sensor module 12, or directly from sensor 10, and sends commands to gas delivery mechanism 40, as well as other components within pneumatic module 30, as required, to control the delivered FiO₂ to the patient.

Operator input 82 includes, inter alia, sensor data thresholds, a desired percentage of FiO₂ and an FiO₂ low threshold, corresponding to the lowest acceptable FiO₂ value. Sensor data 80 include sensor measurements and associated status information, such as, for example, signal quality indicators, etc. In a preferred embodiment, sensor 10 is a pulse oximeter, and sensor data 80 include S_{P}O₂ measurements, perfusion index, signal quality index, measurement artifact indicators, sensor failure data, etc. Operator input 82 correspondingly includes an S_{P}O₂ low threshold, corresponding to the low point of the intended S_{P}O₂ target range, and an S_{P}O₂ high threshold, corresponding to the high point of the intended S_{P}O₂ target range.

Closed-loop FiO₂ control process 90 provides sensor data filtering 92, FiO₂ control 94 and output processing 96. Sensor data filtering 92 receives measurement data representing the oxygen concentration in the patient's bloodstream, associated status information and sensor data thresholds, processes the sensor data, and determines whether the measurement data is valid. In one embodiment, an oxemia state, indicating the level of oxygen concentration in the patient's bloodstream relative to a low range, a normal range and a high range, is determined from the measurement data. FiO₂ control 94 receives the processed sensor data and oxemia state, sensor data thresholds, the desired percentage of FiO₂ and the FiO₂ low threshold, and determines the delivered FiO₂, as well as other operating parameters for pneumatic module 30, such as gas mixture flow rate, delivery pressure, etc. Output processing 96 converts the delivered FiO₂ and operating parameters to specific commands for gas delivery mechanism 40, as well as other pneumatic module 30 components, as required.

In a preferred embodiment, FiO₂ control 94 controls the delivered FiO₂ based on the desired oxygen concentration, the measured oxygen concentration, an FiO₂ baseline and an FiO₂ oxemia state component. The FiO₂ baseline represents the average level of FiO₂ required to maintain the patient in a stable normoxemia state, while the FiO₂ oxemia state component provides for different control algorithms, such as proportional, integral, proportional-integral, etc.

Advantageously, FiO₂ control 94 ensures that the oxygen concentration in the patient's bloodstream does not fall below a low threshold, nor rise above a high threshold, when the sensor data is determined to be invalid. This determination is based not only on the representative oxygen concentration measurements, but also, and importantly, on the status information associated with the sensor measurements. For example, while sensor module 12 may provide a particular measurement value that appears to fall within a normal oxygen concentration range, this data may actually be suspect, as indicated by one or more associated confidence metrics provided by sensor module 12.

In the pulse oximeter embodiment, sensor data filtering 92 receives S_{P}O₂ low and high thresholds, and examines measured S_{P}O₂, perfusion index, signal quality index, measurement artifact indicators, sensor failure data, etc., to determine whether the S_{P}O₂ measurement is valid, and stores one or more seconds of S_{P}O₂ data. The oxemia state is determined from the S_{P}O₂ measurements and the S_{P}O₂ thresholds. In a preferred embodiment, a hypoxemia state (low range) is determined if the S_{P}O₂ measurement is less than the S_{P}O₂ low threshold, a hyperoxemia state (high range) is determined if the S_{P}O₂ measurement is higher than the S_{P}O₂ high threshold, and a normoxemia state (normal range) is determined if the S_{P}O₂ measurement is between the S_{P}O₂ low and high thresholds. While specific values for S_{P}O₂ low and high thresholds will be prescribed by the clinician based on the patient's particular need, these thresholds typically fall within the range of 80% to 100%. For example, the S_{P}O₂ low threshold might be set to 87%, while the S_{P}O₂ high threshold might be set to 93%. The most recent S_{P}O₂ measurement may be used in the determination, or, alternatively, a number of prior S_{P}O₂ measurements may be processed statistically (e.g., median, mean, etc.) and the resultant value used in the determination.

In this embodiment, FiO₂ control 94 receives the processed S_{P}O₂ measurement, perfusion index, signal quality index, etc., and oxemia state, S_{P}O₂ thresholds, the desired percentage of FiO₂ and the FiO₂ low threshold, and calculates the delivered FiO₂ and other operating parameters for pneumatic module 30. While a specific value for FiO₂ low threshold will be prescribed by the clinician based on the patient's particular need, this threshold typically falls within the range of 21% to 100%, such as, for example, 40%. With respect to the FiO₂ low threshold, if the calculated value for the delivered FiO₂ is less than the FiO₂ low threshold, then FiO₂ control 94 sets the delivered FiO₂ to the FiO₂ low threshold value. Similarly, with respect to the S_{P}O₂ thresholds if the measured S_{P}O₂ is below a lower S_{P}O₂ threshold, FiO₂ control 94 increases the calculated value for the delivered FiO₂, and, if the measured S_{P}O₂ is above a higher S_{P}O₂ threshold, FiO₂ control 94 decrease the calculated value for the delivered FiO₂. With respect to the sensor status information, if the perfusion index is less than a perfusion threshold, such as, for example, 0.3%, FiO₂ control 94 sets the delivered FiO₂ to a predetermined value. Similarly, if the signal quality index is less that a signal quality threshold, such as, for example, 0.3, FiO₂ control 94 sets the delivered FiO₂ to a predetermined value and optionally triggers an audio or visual alarm. Similar behavior may be adopted for measurement artifact indicators, sensor failure data, etc.

In a further embodiment, in order to linearize the effect of the control of blood oxygen tension, changes in FiO₂ in the normoxia and hypoxemias states may be calculated from notional oxygen tension. In this embodiment, FiO₂ control 94 first applies a transformation to the S_{P}O₂ values to normalize frequency distribution, and then applies one or more linear filters to the transformed S_{P}O₂ values. One such transformation is an inverse transform of the oxyhemoglobin saturation curve.

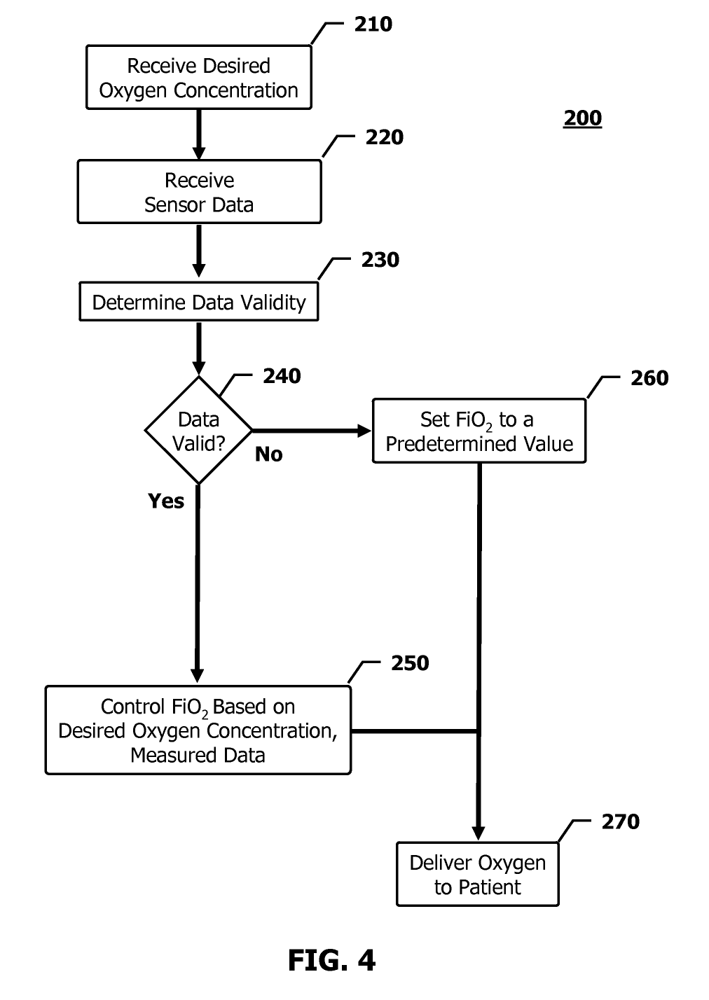

FIG. 4 is flow chart depicting a method 200 for automatically delivering oxygen to a patient, in accordance with an embodiment of the present invention.

A desired oxygen concentration is first received (210) from a user. As discussed above, the user may input the desired oxygen concentration, such as, for example, the desired percentage of FiO₂, using input device(s) 26 and display 24.

Sensor data are received (220) from sensor module 12, or directly from sensor 10, through sensor interface 14. As discussed above, sensor data include a measurement of the amount of oxygen in the bloodstream of the patient and status information associated with the measurement, such as, for example, saturation of peripheral oxygen measurements, arterial oxygen partial pressure measurements, dissolved oxygen in the blood measurements, a perfusion index, a signal quality index, measurement artifacts, sensor status, etc.

The validity of the measured data is then determined (230) based on the value of the measured data and the status information. As discussed above, sensor data filtering 92 receives measurement data representing the oxygen concentration in the patient's bloodstream, associated status information and sensor data thresholds, processes the sensor data, and determines whether the measurement data are valid.

If the measured data are determined to be valid (240), then the FiO₂ delivered to the patient is controlled (250) based on the desired oxygen concentration and the measured data. As discussed above, FiO₂ control 94 receives the processed sensor data, sensor data thresholds, and the desired percentage of FiO₂ and controls the delivered FiO₂ based on the desired percentage of FiO₂ and the measured oxygen concentration.

On the other hand, if the measured data are not determined to be valid (240), FiO₂ control 94 sets (260) the FiO₂ delivered to the patient to a predetermined value.

The gas mixture, with the determined FiO₂ percentage of oxygen, is then delivered (270) to the patient.

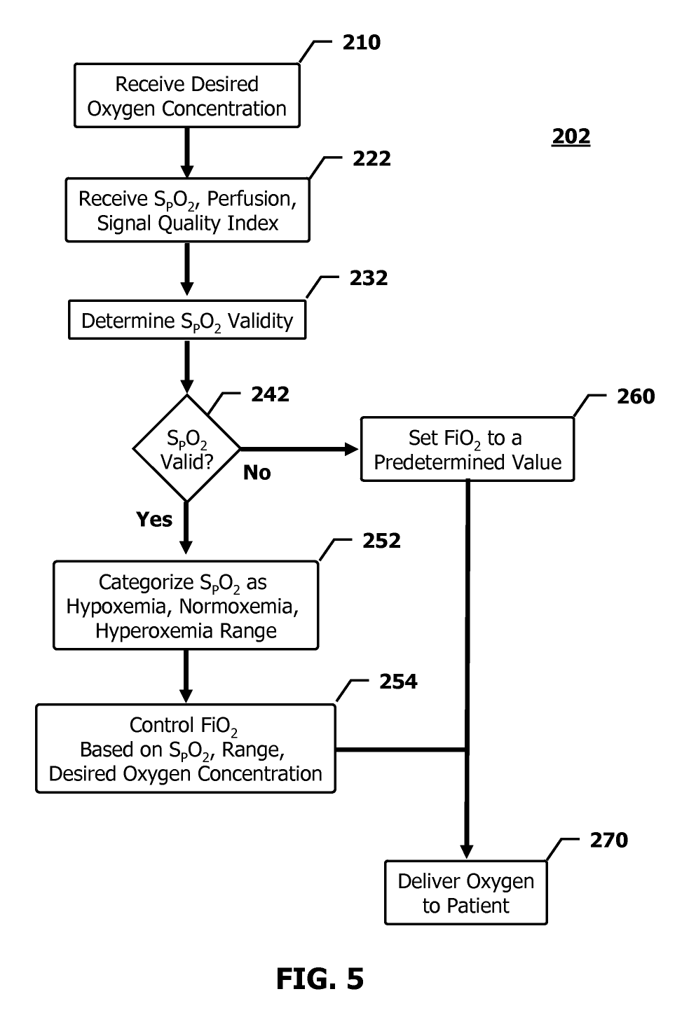

FIG. 5 is flow chart depicting a method 202 for automatically delivering a breathing gas mixture with a calculated percentage of oxygen to a patient, in accordance with another embodiment of the present invention.

A desired oxygen concentration is first received (210) from a user. As discussed above, the user may input the desired oxygen concentration, such as, for example, the desired percentage of FiO₂, using input device(s) 26 and display 24.

Pulse oximeter data are received (222) from the pulse oximeter module, or directly from the pulse oximeter, through sensor interface 14. As discussed above, pulse oximeter data include a measurement of the saturation of peripheral oxygen, S_{P}O₂, in the bloodstream of the patient, a perfusion index, a signal quality index, and, optionally, an indication of measurement artifacts, pulse oximeter status, etc.

The validity of the measured S_{P}O₂ is then determined (232) based on the value of the measured S_{P}O₂ and at least one of the perfusion index and the signal quality index, and, optionally, the measurement artifact indication(s), the pulse oximeter status, etc. As discussed above, sensor data filtering 92 receives the measured S_{P}O₂, perfusion index, signal quality index, etc., and S_{P}O₂ data thresholds, processes the data, and determines whether the measured S_{P}O₂ is valid. Sensor data filtering 92 also determines the oxemia state based on the measured S_{P}O₂.

If the measured S_{P}O₂ is determined to be valid (242), then the measured S_{P}O₂ is categorized within a hypoxemia, normoxemia or hyperoxemia range, and the FiO₂ delivered to the patient is controlled (254) based on the desired percentage of FiO₂, the measured S_{P}O₂, and the respective range. As discussed above, FiO₂ control 94 receives the oxemia state, the FiO₂ threshold, the processed S_{P}O₂, the S_{P}O₂ thresholds, and the desired percentage of FiO₂ and controls the delivered FiO₂ based on the desired percentage of FiO₂, the measured S_{P}O₂ and the respective range. FiO₂ control 94 ensures that the delivered FiO₂ to not less than the FiO₂ threshold, increases the delivered FiO₂ if the measured S_{P}O₂ is below the lower S_{P}O₂ threshold, and decreases the FiO2 if the measured S_{P}O₂ is above the upper S_{P}O₂ threshold.

On the other hand, if the measured S_{P}O₂ is not determined to be valid (242), FiO₂ control 94 sets (260) the FiO₂ delivered to the patient to a predetermined value.

The oxygen is then delivered (270) to the patient.

The many features and advantages of the invention are apparent from the detailed specification, and, thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and, accordingly, all suitable modifications and equivalents may be resorted to that fall within the scope of the invention.

What is claimed is:

1. An automated oxygen delivery system, comprising:
a sensor to measure an amount of oxygen in a bloodstream of a patient;
a pneumatics subsystem, including:
an oxygen inlet, an air inlet, a gas mixture outlet, and
a gas delivery mechanism, coupled to the oxygen inlet, the air inlet and the gas mixture outlet, to blend oxygen and air to form a gas mixture having a delivered oxygen concentration, and to deliver the gas mixture to the patient; and
a control subsystem, coupled to the sensor and the pneumatics subsystem, including:
an input device to receive a desired concentration of oxygen in the bloodstream of the patient,
a sensor interface to receive measurement data and status information associated with the measurement data from the sensor,
a pneumatics subsystem interface to send commands to, and receive data from, the pneumatics subsystem, and
a processor, coupled to the input device, the sensor interface and the pneumatics subsystem interface, to control the delivered oxygen concentration based on the desired oxygen concentration, the measurement data and the status information.

2. The automated oxygen delivery system of claim 1, wherein the air inlet receives a mixture of breathable gases.

3. The automated oxygen delivery system of claim 1, wherein the gas delivery mechanism controls a flow rate and a delivery pressure of the gas mixture.

4. The automated oxygen delivery system of claim 1, wherein the delivered oxygen concentration is expressed as a fraction of inspired oxygen, FiO₂.

5. The automated oxygen delivery system of claim 4, wherein the delivered FiO₂ is not less than an FiO₂ threshold.

6. The automated oxygen delivery system of claim 4, wherein the sensor is a pulse oximeter, and the sensor data include a saturation of peripheral oxygen measurement, S_{P}O₂, a perfusion index and a signal quality index.

7. The automated oxygen delivery system of claim 6, wherein the processor controls the delivered oxygen concentration based on the S_{P}O₂, the perfusion index and the signal quality index.

8. The automated oxygen delivery system of claim 7, wherein the processor increases the FiO₂ if the measured S_{P}O₂ is below a lower S_{P}O₂ threshold, and decreases the delivered FiO₂ if the measured S_{P}O₂ is above an upper S_{P}O₂ threshold.

9. The automated oxygen delivery system of claim 7, wherein the processor sets the FiO₂ to a predetermined value if the perfusion index is less than a perfusion threshold.

10. The automated oxygen delivery system of claim 7, wherein the processor sets the FiO₂ to a predetermined value if the signal quality index is less than a signal quality threshold.

11. The automated oxygen delivery system of claim 10, wherein the processor initiates at least one of an audible alarm and a visual alarm if the signal quality index is less than a signal quality threshold.

12. The automated oxygen delivery system of claim 4, wherein the sensor is a transcutaneous gas tension sensor, and the sensor data include an arterial oxygen partial pressure measurement, PtcO₂, and an arterial carbon dioxide partial pressure measurement, PtcCO₂.

13. The automated oxygen delivery system of claim 4, wherein the sensor is an invasive catheter blood analyzer, and the sensor data include a dissolved oxygen in the blood measurement, pO₂, a dissolved carbon dioxide in the blood measurement, pCO₂, a blood acidity pH measurement, and a blood temperature measurement.

14. An automated oxygen delivery system, comprising:
a pulse oximeter sensor to measure saturation of peripheral oxygen, S_{P}O₂, in a bloodstream of a patient;
a pneumatics subsystem, including:
an oxygen inlet, an air inlet, a gas mixture outlet, and
a gas delivery mechanism, coupled to the oxygen inlet, the air inlet and the gas mixture outlet, to blend oxygen and air to form a gas mixture having a delivered fraction of inspired oxygen, FiO₂, and to deliver the gas mixture to the patient; and
a control subsystem, coupled to the sensor and the pneumatics subsystem, including:
an input device to receive a desired concentration of oxygen in the bloodstream of the patient,
a sensor interface to receive S_{P}O₂ measurements and status information associated with the measurement from the sensor, the status information including a perfusion index and a signal quality index,
a pneumatics subsystem interface to send commands to, and receive data from, the pneumatics subsystem, and
a processor, coupled to the input device, the sensor interface and the pneumatics subsystem interface, to control the FiO₂ based on the desired oxygen concentration, the S_{P}O₂, the perfusion index and the signal quality index, and to set the FiO₂ to a predetermined value if the perfusion index value is less than a perfusion threshold or the signal quality index is less than a signal quality threshold.

15. The automated oxygen delivery system of claim 14, wherein the air inlet receives a mixture of breathable gases.

16. The automated oxygen delivery system of claim 14, wherein the gas delivery mechanism controls a flow rate and a delivery pressure of the gas mixture.

17. The automated oxygen delivery system of claim 14, wherein the FiO₂ is not less than an FiO₂ threshold.

18. The automated oxygen delivery system of claim 14, wherein the processor increases the FiO₂ if the measured S_{P}O₂ is below a lower S_{P}O₂ threshold, and decreases the FiO₂ if the measured S_{P}O₂ is above an upper S_{P}O₂ threshold.

19. The automated oxygen delivery system of claim 14, wherein the perfusion index is a fractional variation in the optical absorption of oxygenated red blood cells between the systole and diastole periods of an arterial pulse.

20. The automated oxygen delivery system of claim 14, wherein the signal quality index provides a confidence metric for the S_{P}O₂.

21. The automated oxygen delivery system of claim 20, wherein the signal quality index is based on variations in the optical absorption of oxygenated red blood cells.

22. A system for automatically delivering oxygen to a patient, comprising:
a means for measuring an amount of oxygen in a bloodstream of a patient;
a pneumatics subsystem, including:
an oxygen inlet, an air inlet, a gas mixture outlet,
a means for blending oxygen and air to form a gas mixture having a delivered oxygen concentration, and
a means for delivering the gas mixture to the patient; and
a control subsystem, coupled to the means for measuring the amount of oxygen and the pneumatics subsystem, including:
an input device to receive a desired concentration of oxygen in the bloodstream of the patient;
a first interface to receive measurement data and status information associated with the measurement data from the means for measuring the amount of oxygen,
a second interface to send commands to, and receive data from, the pneumatics subsystem, and
a processor, coupled to the first interface and the second interface, to control the delivered oxygen concentration based on the desired oxygen concentration, the measurement data and the status data.
